(19) United States
(12) Patent Application Publication
(10) Pub. No.: US 2009/0305401 A1
(43) Pub. Date: Dec. 10, 2009

(54) PLASMA-FREE PLATELET LYSATE FOR USE AS A SUPPLEMENT IN CELL CULTURES AND FOR THE PREPARATION OF CELL THERAPEUTICS

(76) Inventors: Dirk Strunk, Kumberg (AT); Katharina Schall-Moser, Graz (AT); Eva Rohde, Graz (AT)

Correspondence Address: K&L Gates LLP, P.O. Box 1135, CHICAGO, IL, 60690, US

(21) Appl. No.: 12/441,870

(22) PCT Filed: Sep. 17, 2007
(86) PCT No.: PCT/EP07/59796

§ 371 Date: Aug. 10, 2009

(30) Foreign Application Priority Data
Sep. 18, 2006 (EP) 06120857.5

(51) Int. Cl. C12N 5/06

(52) U.S. Cl. 435/325; 435/404; 435/407

(57) ABSTRACT

The present invention provides a cell culture medium supplement comprising plasma-free platelet lysate and medium supplemented with this supplement. The present invention further provides a method for preparing the supplement comprising the steps of (a) preparing platelet rich plasma; (b) removing the plasma; and (c) lysing the platelets.

The present invention provides a cell culture medium supplement comprising plasma-free platelet lysate and medium supplemented with this supplement. The present invention further provides a method for preparing the supplement comprising the steps of (a) preparing platelet rich plasma; (b) removing the plasma; and (c) lysing the platelets. Tie present invention is also concerned with the use of this culture supplement for growing cells and particularly stem cells.

In recent years, the stem cell therapy has become increasingly popular because it has the potential to improve organ regeneration in a large spectrum of diseases, e.g. after ischemic, metabolic or toxic organ injury. Furthermore, it has been shown that mesenchymal stem cells (MSCs) have an immunomodulatory effect which can be used for avoiding graft rejections after organ transplantations, graft versus host disease after stem cell transplantations and autoimmune diseases (reviewed in Rasmusson (2006) Exp. Cell Res. 312(12): 2169-2179; Krampera et al. (2006) Curr. Opin. Pharmacol. 6(4): 435-441). Furthermore, MSCs are also interesting for a cell-based regenerative medicine, as they can be stimulated to differentiate towards lineages of the mesenchymal tissue, including bone, cartilage, fat, muscle, tendon and marrow stroma. MSCs are already employed in preclinical studies to regenerate bone in massive bone defects which the body cannot naturally repair.

Although stem cells exist in low numbers in almost all organs, selected types of stem cells need to be expanded ex vivo to produce the required quantity of stem cells for clinical application into patients. For the expansion of MSCs, most often Fetal Bovine Serum (FBS) is used. However, the use of FBS bears the risk of transmission of known and unknown pathogens. Known pathogens are e.g. prions transmitted in bovine spongiforme encephalopathy. Therefore, the use of FBS for clinical stem cell culture is prohibited in Germany since 2001 and is expected to be prohibited in the whole EU and the USA in the near future.

An alternative for using FBS might be the use of lysates of human platelet-rich plasma (PRP).

In this respect, it has been shown that a platelet-released supernatant increases the proliferation of bone cells which is at least partially due to the action of multiple platelet derived growth factors which are released from the platelets after activation by agonists such as thrombin or by physical influences such as freezing and thawing (Zimmermann et al. (2001) Transfusion 41: 1217-1224).

The dose-dependent proliferation of MSCs upon treatment with platelet lysate was also observed (Lucarelli et al (2003) Biomaterials 24: 3095-3100). The treatment of the cells with platelet lysate did not reduce their capability to differentiate along the chondrogenic and osteogenic lineage.

Furthermore, it was shown that MSCs cultured in the presence of platelet lysate proliferate even faster than the cells cultured in FBS-supplemented medium. Also in this case MSCs cultured in the presence of a platelet lysate maintained their osteogenic, chondrogenic, and adipogeneic differentiation properties and retained their immunomodulatory activity (Doucet et al. (2005) J. Cell Physiol. 205: 228-236).

However, the procedures producing PRP are highly variable and depend on the supplier. Furthermore, there is the risk of disease transmission by human plasma. For example, it has been shown that transfusion-related acute lung injury (TRALI) may be due to the transfusion of plasma-containing blood products. This might be due to the passive transfer of neutrophil or HLA antibodies from the donor or the transfusion of biologically active lipids from older, cellular blood products (Looney et al. (2004) Chest 126: 249-258). Moreover, plasma transfusion may lead to the infection with pathogens which cannot be removed by the conventional viral reduction processes during plasma fractionation, such as non-lipid coated viruses (Ludlam et al (2006) Lancet 367: 252-261).

Additionally, incompatibilities due to blood group-related antibodies (isoagglutinins) present in plasma preparations necessitate the selection of blood group-compatible platelet lysate products or the use of blood group AB plasma which is devoid of anti AB antibodies. This dramatically limits the availability of starting material for platelet lysate production because blood group AB comprises less than 5% of all donors (compared to blood group 0 with a frequencyof approximately 45% in the Caucasian population).

Platelet preparations with reduced plasma content have been used for transfusion. For example, a platelet storage solution called T-SOL was developed which consists of sodium chloride, sodium citrate, and sodium acetate (Hogmann et al. (1997) Transfus. Sci. 18: 3-13, Van Rhenen et al. (2004) Transfusion Medicine 14: 289-295).

However, the plasma could not be completely substituted by the solution, but 35% of plasma had to be present in the platelet storage solution to achieve an appropriate response after transfusion. In a study investigating the cryopreservation of leukocyte-reduced platelet concentrates, different compositions of the cryopreservation medium were used (Dijkstra-Tiekstra et al (2003) Vox Sanguinis 85:276-282). It could be shown that a minimum of 50% plasma in the cryopreserved leukocyte-reduced platelet concentrates is necessary to maintain an acceptable in vitro quality of platelets up to 24 hours after thawing.

Recently it was shown that a preservative solution comprising a preservative such as trehalose, water and protein (e.g. albumin) is suitable for the cryopreservation of platelets when loaded into the platelets (WO 2005/020893). However, it is unlikely that such a solution can also be used for substituting plasma in the platelet lysate which is intended to be used as a cell culture medium supplement, since the trehalose alters the osmolarity of the solution.

Therefore, there is still a great need inter alia for a cell culture medium supplement which is capable of supporting the growth of cells, in particular MSCs, comprising a platelet lysate which is plasma-free.

### OBJECT AND SUMMARY OF THE INVENTION

It is an object of the present invention to provide a cell culture supplement which can be used for efficiently culturing cells and which is substantially devoid of pathogens.

It is a further object of the present invention to provide a method for preparing a cell culture supplement which can be used for efficiently culturing cells and which is substantially devoid of pathogens.

It is yet another object to provide a cell culture medium supplement for culturing stem cells and progenitor cells.

These and further objects of the invention, as will become apparent from the description, are attained by the subject-matter of the independent claims.

Further embodiments of the invention are defined by the dependent claims.

According to one aspect of the invention, a cell culture medium supplement is provided which comprises a plasma-free platelet lysate.

In a preferred embodiment of the present invention, the supplement further comprises a substance selected from the group consisting of albumin, dextran and hydroxyethyl starch. More preferably, the supplement comprises albumin, and most preferably it comprises human serum albumin. In a particularly preferred embodiment, recombinant versions of albumin may be used in the culture supplement. Such preparations are e.g. available under the trade names Albucult™ and Recombunin™ from Novozymes Delta Ltd (Nottingham, UK).

In a further preferred embodiment of the invention, the concentration of albumin in the cell culture medium supplement is 2-7% v/v, more preferably it is 5% v/v.

In a further embodiment, the cell culture medium supplement additionally comprises acid citrate dextrose or citrate phosphate dextrose.

In a further embodiment of the present invention, the plasma-free platelet lysate is prepared from a solution with a platelet concentration of 1×10⁸-5×10⁹ per ml, preferably with a concentration of 1-2×10⁹ per ml.

The plasma-free platelet lysate may be prepared from apheresis platelet concentrates or buffy coat units, preferably it is prepared from buffy coat units.

In a further aspect of the present invention, a cell culture medium is provided which is supplemented with the inventive supplement comprising plasma-free platelet lysate.

In a preferred embodiment, the supplement is present in the medium in a concentration of 1-20% v/v, preferably in a concentration of 2% to 18% v/v and more preferably in a concentration of 4% to 16% v/v. A particularly preferred concentration is approximately 10% V/V.

In a further preferred embodiment, the medium is a-MEM, which is Modified Eagle Medium being available from e.g. Invitrogen GmbH (Karlsruhe, Germany)

In a further aspect of the present invention, a method for preparing a supplement comprising plasma-free platelet lysate is provided, comprising the following steps:
a) preparing platelet-rich plasma;
b) removing the plasma; and
c) lysing the platelets.

In a preferred embodiment of the method of the present invention, the method further comprises the step of adding a substance selected from the group consisting of albumin, dextran and hydroxyethyl starch before or after lysing the platelets. More preferably, the substance is albumin and most preferably it is human serum albumin. In a particularly preferred embodiment, recombinant versions of albumin may be used in the culture supplement. Such preparations are e.g. available under the trade names Albucult™ and Recombunin™ from Novozymes Delta Ltd (Nottingham, UK).

According to a further embodiment of the method of the present invention, the albumin is added to the medium to yield a final concentration of 2-7% v/v, preferably of 5% v/v.

In a preferred embodiment of the present invention the platelet-rich plasma is prepared from buffy coat units.

In a further preferred embodiment of the present invention, the plasma is removed by centrifugation.

In still another embodiment of the present invention, the platelets are lysed by freezing and thawing them.

In a further preferred embodiment of the present invention, the concentration of the platelets before lysis is 1×10⁸-5×10⁹ per ml, preferably 1-2×10⁹ per ml.

A further aspect of the present invention relates to a method for preparing a medium, comprising the step of mixing a cell culture medium with a supplement comprising plasma-free platelet lysate.

Still another aspect of the present invention relates to a method of culturing cells, wherein the cells are cultured in a medium supplemented with the supplement comprising plasma-free platelet lysate.

In a preferred embodiment, the cultured cells are stem cells or progenitor cells. Particularly preferred the cultured cells are MSCs.

In a further preferred embodiment, the cells are cultured for at least 10 days in the medium comprising a cell culture medium supplement comprising plasma-free platelet lysate.

Another aspect of the present invention relates to the use of the cell culture medium supplement comprising plasma-free platelet lysate for supplementing a culture medium.

Still another aspect of the present invention relates to the use of a medium supplemented with the cell culture medium supplement comprising plasma-free platelet lysate for culturing cells.

### DESCRIPTION OF THE DRAWINGS

FIG. 1 shows the cell numbers and fold increase of the cell numbers of MSCs cultured in a-MEM supplemented either with 10% plasma-free platelet lysate in 5% human albumin (PL-HA), 10% platelet lysate (PL), FBS or EBMT clinical study FBS on day 12. Cells were initially seeded in a density of 50-100 cells/cm².

FIG. 2 shows microphotographs of MSCs cultured in media containing the different medium supplements described in FIG. 1. The photograph was taken on day 12.

FIGS. 3a and b show the results of cytokine measurements in media supplemented with PL-HA or PL before (day 0) and after culture (day 12). The cytokines measured are indicated.

FIGS. 4a, b and c show the result of growth factor measurements in media supplemented with PL-HA or PL before (day 0) and after culture (day 12). The growth factors measured are indicated.

### DETAILED DESCRIPTION OF EXEMPLARY EMBODIMENTS

Before describing in detail exemplary embodiments of the present invention, the following definitions are given.

The term “approximately” describes a deviation of the indicated value by at least 10%, preferably by at least 5% and most preferably by at least 1%.

The term “cell culture” refers to the maintenance and propagation of cells and preferably animal (including humanderived cells) in vitro. The cells may include stem cells and progenitor cells as defined below. Ideally the cultured cells do not differentiate and do not form organized tissues, but undergo mitosis synchronously.

“Cell culture medium” is used for the maintenance of cells in culture in vitro. For some cell types, the medium may also be sufficient to support the proliferation of the cells in culture. A medium according to the present invention provides nutrients such as energy sources, amino acids and anorganic ions. Additionally, it may contain a dye like phenol red, sodium pyruvate, several vitamins, free fatty acids and trace elements.

The term “cell culture medium supplement” within the meaning of the present invention refers to a medium additive which is added to the medium to stimulate the proliferation of the cells. Usually this supplement will contain one or more growth factors which are responsible for the stimulation of proliferation. The term “supplement” is not intended to comprise medium additives which are added to the medium for the purpose of freezing the cells. In addition to the cell culture medium supplement of the present invention, other compounds such as honnones, glutaine, ribonucleotides, desoxyribonucleotides and antibiotics, etc. may be added to the medium.

The term “maintenance of cells” is intended to mean that the cell number remains substantially unchanged, i.e. neither increases nor decreases.

The term “proliferation of cells” is intended to mean the multiplication of cells thereby leading to an increase in the cell number. The proliferation of cells may be detected by any suitable method. The easiest way to measure proliferation is to seed the cells in a specific, predetermined density and to count the cell number at different time points after seeding. Another way of measuring the proliferation of cells is a [³H]-thymidine incorporation assay which involves the addition of [³H]-thymidine to the cells, incubating them for a specific time, lysing the cells and counting the incorporation in a scintillation counter. Commercially available kits like the tetrazolium assay (MTT, Sigma) may also be used for measuring proliferation.

The term “growth factor” is intended to comprise proteins which stimulate proliferation of cells by binding to a specific receptor. Usually, growth factors only act on specific cell types which express the respective receptor. Examples of growth factors are epidermal growth factor (EGF), nerve growth factor (NGF), platelet derived growth factor (PDGF), fibroblast growth factor (FGF), vascular endothelial growth factor (VEGF), bone morphogenetic proteins (BMP), colony stimulating factors (CSF) etc.

The term “plasma” refers to the fluid component of the blood in which the particulate material is suspended. The plasma makes up about 55% of the whole blood and contains proteins such as albumins, globulins and fibrinogen, water, ions, nutrients and platelets. However, the plasma does not contain blood cells such as erythrocytes and leukocytes.

The term “plasma-free” means that the lysate contains less than 10%, preferably less than 9, 8, 7, 6 or 5%, more preferably less than 4, 3, 2, 1%, even more preferably less than 0.9, 0.8, 0.7, 0.6 or 0.5% and most preferably less than 0.4, 0.3, 0.2, 0.1% of plasma, compared to a lysate prepared in plasma and/or to a platelet solution before removal of the plasma.

The residual amount of plasma present in the lysate may be determined by detecting plasma components such as globulins or fibrinogen and comparing the amount of one or more of these proteins in the plasma-free lysate with a lysate prepared in plasma and/or with the solution before the plasma has been removed.

The proteinaceous plasma contents may be detected by any suitable method like Western Blotting, immunofluorescent labeling, Northern Blotting, RT-PCR or other methods known to the skilled person. Alternatively or additionally, the efficiency of plasma removal can be measured by mixing the plasma with a predetermined amount of an exogenous agent like inactivated viruses and determining the virus titer before and after removing the plasma. The concentration of the protein and/or the virus titer after removing the plasma should be less than 10%, preferably less than 8, 7, 6 or 5%, more preferably less than 4, 3, 2, 1%, even more preferably less than 0.9, 0.8, 0.7, 0.6 or 0.5% and most preferably less than 0.4, 0.3, 0.2, 0.1% of the concentration and/or the titer before removing the plasma.

Platelets originate as cell fragments or “minicells” (without nuclear DNA) from megakaryocytes of the bone marrow. Platelets are characteristically activated at sites of injury where they create a physical barrier to limit blood loss and accelerate the generation of thrombin to intensify the coagulation process. Additionally, they are involved in wound healing and repair of mineralized tissue (Gentry (1992) Journal of Comparative Pathology 107: 243-270; Barnes et al (1999) Journal of Bone Mineral Research 14: 1805-1815). This latter function is mediated by the release of growth factors which are chemoattractants for inesenchymal cells of the external soft tissue and the bone marrow (Barnes et al (1999) Journal of Bone Mineral Research 14: 1805-1815).

The term “lysate” refers to the product of the lysis of cells, i.e. the product of disrupting the cellular integrity which leads to the release of the molecules which are normally present within the cells into the solution. The cellular integrity is disrupted by at least partially destroying the cell membrane.

Preferably, the plasma-free platelet lysate of the present invention further comprises a substance selected from the group consisting of albumin, dextran and hydroxyethyl starch.

Albumin is the major protein component of the blood plasma making up up to 60-80% of the protein content. It is soluable in water and in dilute salt solutions. It is also contained in body fluids other than blood, e.g. in milk (=lactalbumin) and eggs (=ovalbumin). The albumin used should preferably be from the same species as the platelets, meaning that human serum albumin should be used together with human platelets. In a particularly preferred embodiment, recombinant versions of albumin may be used in the culture supplement. Such preparations are e.g. available under the trade names Albucult™ and Recombunin™ from Novozymes Delta Ltd (Nottingham, UK).

Dextran is a complex branched polysaccharide made of many glucose molecules joined into chains of different length. The straight chain consists of a 1→6 glucosidic linkages between glucose molecules, while branches begin from a 1→3 linkages. Dextrans are available in multiple molecular weights ranging from 10.000 Dalton to 150.000 Dalton. Preferably the Dextran has a molecular weight between 30.000 and 50.000 Dalton, most preferably it has a molecular of about 40.000 Dalton which is also referred to as Dextran 40.

Hydroxyethyl starch refers to starch derivatives which are substituted with a hydroxyethyl group. It is derived from a waxy starch composed almost entirely of amylopectin with hydroxyethylether groups introduced into the alpha (1→4) linked glucose units. Preferably, hydroxyethyl starch has a mean molecular weight of 1-300 kDa, more preferably it has a mean molecular weight of 5-200 kDa. Different types of hydroxyethyl starches may be further characterized by their degree of substitution and the site of hydroxyethylation on the glucose molecule. Usually hydroxyethyl starch is used in a concentration of about 6%.

Preferably, the substance is albumin and more preferably it is (recombinant) human serum albumin.

Human serum albumin can be obtained from suppliers such as Sanquin Plasma Products, Mediatech, Inc., SeraCare, Sigma Aldrich, Sera Laboratories International, Valley Biomedical, Baxter and Behring.

The concentration of albumin in the cell culture supplement is between 1-10% v/v, preferably it is between 2-7% v/v, more preferably it is between 3-6% v/v and most preferably it is approximately 5% v/v.

“Acid Citrate Dextrose (ACD)” is an important blood anticoagulant. At present, there are two widely used forms of acid citrate dextrose. Solution A comprises 22.0 g/l tlisodium citrate, 8.0 g/l citric acid and 24.5 g/l dextrose. Solution B comprises 13.2 g/l trisodium citrate, 4.8 g/l citric acid and 14.7 g/l dextrose. Preferably, solution A is used. ACD is used for blood bank studies, HLA phenotyping, flow cytometry testing, tissue typing, DNA and paternity testing and blood preservative.

The concentration of ACD within the supplement is 5-15% v/v, preferably 6-13% v/v, more preferably 8-12% v/v and most preferably approximately 10% v/v.

Instead of ACD, the anticoagulant citrate phosphate dextrose (CPD) which comprises 26.3 g/l sodium dihydrate, 3.27 g/l citric acid monohydrate, 25.5 g/l glucose monohydrate and 2.51 g/l sodium dihydrogenphosphate dihydrate may be used in the supplement of the present invention.

The concentration of CPD within the supplement is 5-20% v/v, preferably 6-18% v/v, more preferably 8-12% v/v and most preferably approximately 10% v/v.

The plasma-free platelet lysate is prepared from a solution that has a platelet concentration of 1×10⁸ to 5×10⁹ per ml, preferably of 5×10⁸ of 3×10⁹ per ml and most preferably of approximately 1-2×10⁹ per ml.

The cell culture medium supplement according to the present invention may be used to supplement a cell culture medium. The concentration of the supplement in the medium is between 1-20% v/v, preferably between 5-18% v/v, more preferably between 8-12% v/v and most preferably approximately 10% v/v.

As the supplement comprising the plasma-free platelet lysate provides growth factors which are necessary for the growth of cells and which are released from the platelets upon lysis of the cells, the concentration of the supplement in the medium and the platelet concentration from which the supplement is prepared are linked with each other. Therefore a lower concentration of supplement in the medium can be used, if the lysate is prepared from a solution with a high platelet concentration and accordingly, a higher concentration of a supplement is necessary, if the lysate is prepared from a solution with a low platelet concentration.

In particular, this means that if the lysate is prepared from a platelet solution with a concentration of 5×10⁹ per ml, a supplement concentration in the medium of 1-2% may be sufficient. Accordingly, if the lysate is prepared from a platelet solution with a concentration of 1×10⁸ per ml, a supplement concentration in the medium of 20% v/v may be necessary.

The skilled person knows methods how to determine the optimal combination of the concentration of the platelets in the solution from which the lysate is prepared and the concentration of the supplement in the medium. For example, one can prepare a lysate from solutions with different concentrations of platelets and add these lysates in different concentrations to the medium. Afterwards, the proliferation rate of the cells is compared and the combination is chosen which gives the highest proliferation rate. The proliferation rate may be determined by seeding a defined number of cells which is the same for each condition, counting the cells at different time points after seeding and comparing the growth rate from the different conditions. Such proliferation experiments are within the routine work of the skilled artisan.

The type of medium which is supplemented with the cell culture medium supplement of the present invention depends on the type of cells which is to be cultured in the cell culture medium. The skilled person knows how to select the medium which is suitable for culturing a particular cell type. For culturing MSCs, for example, alpha-MEM, DMEM, DMEM/F12, MesenCult™ (StemCell) and MSCGM (Cambrex) are suitable. Further suitable media are IMDM, Optimem, DMEM/LG/L-G, DMEM/HG/L-G, DMEM/HiG/GL, DMEM/LG/GL, alpha-MEM/L-G, alpha-MEM/GL (Life Technologies; Sotiropoulou et al. (2006) Stem Cells 24(5): 1409-1410) and Poietics Human Mesenchymal Stem Cell Medium (M2; PT-3001, Cambrex; Wagner et al. (2005) Exp. Hem. 33(11): 1402-1416).

Preferably, the medium for MSCs is alpha-MEM. This medium is characterized by the presence of lipoic acid, sodium pyruvate, ascorbic acid and vitamin B12. The medium may be purchased from companies such as Cambrex, Invitrogen, Sigma-Aldrich and Stem Cell Technologies.

Another embodiment of the present invention refers to a method for preparing the cell culture medium supplement according to the present invention, comprising the following steps:
(a) preparing platelet-rich plasma;
(b) removing the plasma; and
(c) lysing the platelets.

In a preferred embodiment of the present invention, the method further comprises the step of adding a substance selected from the group consisting of albumin, dextran and hydroxyethyl starch before or after lysing the platelets. As regards the preferred types of albumin, dextran and hydroxyethyl starch and the preferred amounts thereof, reference is made to the above description.

The term “platelet-rich plasma (PRP)” refers to a concentration of platelets in a carrier which concentration is above that of platelets normally found in blood. For example, the platelet concentration may be 2 times, 5 times, 10 times, 100 times or more of the normal concentration in blood.

PRP may comprise blood components other than platelets. It may be 50% or more, 75% or more, 80% or more, 95% or more, 99% or more platelets. The non-platelet components may be plasma, white blood cells and/or any blood component. PRP may be obtained using autologous, allogeneic, or pool sources of platelets and/or plasma. Preferably it is obtained from autologous plasma. It may be formed from a variety of animal sources, including human sources. Preferably it is obtained from human sources. PRP may be prepared in different ways which are summarized in Zimmennann et al. (2001) Transfusion 41: 1217-1224.

One possibility is to prepare PRP by apheresis which is the medical technology in which the blood of a donor or patient is passed through an apparatus that separates out one particular constituent and returns the remainder to the circulation of the donor or patient. A blood cell separator which is set and primed according to the manufacturer's instructions is programmed to collect a specific amount of platelets in a defined volume. On the basis of entered donor data and the ratio of whole blood to ACD, the on-board processor calculates the blood flow rate and the volume to be processed. One example for a blood cell separator is the Cobe Spectra® Apheresis System (Gambro BCT) with the so-called Leukocyte Reduction System (LRS).

Another possibility to prepare PRP is by the tube method which involves the collection of whole blood in tubes containing ACD and the centrifugation of these tubes at high speed. Afterwards, the supernatant is collected and centrifuged at lower speed. The then resulting supernatant is removed and the pellet consists of platelets which are resuspended in plasma. Tubes and needles which are useful for this method are for example supplied by the company Curasan, Kleinostheim, Germany.

A third and particularly preferred method of preparing PRP is the preparation from buffy coat units. “Buffy coat” is the fraction of a centrifuged blood sample that contains white blood cells and platelets. After centrifugation, one can distinguish a layer of clear fluid (the plasma), a layer of red fluid containing most of the erythrocytes and a thin layer in between which represents the buffy coat. A buffy coat can for example be prepared by centrifugation of whole blood at 3.000×g for 13 minutes. Afterwards, PRP can be prepared by centrifugation of the resuspended buffy coat for 5 minutes at 400×g. The white blood cells which may be present in the buffy coat may be separated from the platelets by any suitable method. For example, they can be removed by filtration with filters such as Imuflex® (Terumo), LCR5 (Macopharma) and Autostop™ BC filter (Pall).

Additionally, there are different commercially available systems for the preparation of PRP like Vivostat PRF Preparation Kit®, PCCS Platelet Concentrate Collection System®, Harvest® SmartPReP 2 APC 60 Process and Fibrinet® Autologous Fibrin and Platelet System. These systems are compared in Leitner et al. (2006) Vox Sanguinis 91: 135-139.

The plasma can be removed from the platelets by any method which is suitable to separate the platelets from plasma components such as proteins and ions. Exemplary methods include centrifugation, dialysis or depth filtration.

Preferably the plasma is removed by centrifugation. The centrifugation can comprise one or more centrifugation steps with intermediate washing of the cells, for example with medium or suitable buffers such as phosphate-buffered saline (PBS) or HEPES-buffered saline (HBS). Of course, the above-mentioned separation methods may also be combined.

The cells are centrifuged at a velocity of 1500 to 3000 g, preferably of 1800 to 2800 g, more preferably of 2000 to 2600 g and most preferably of around 2500 g. The cells are centrifuged for 8 to 15 minutes, preferably for 10 minutes.

The platelets may be lysed in any manner which is suitable to open the platelets and/or allow the interior of the cell to escape. Suitable lysis treatments may be with an energy wave (e.g. ultrasound), temperature (heating/cooling-freezing/thawing), osmotic stress, detergent treatment or thrombin treatment.

If the platelets are lysed by thrombin treatment, approximately 1 to 10 units of thrombin per ml platelet solution containing from 1×10⁶ to 1×10⁹ platelets are used. The thrombin treatment may be combined with calcium treatment of the cells. Suitable lysis techniques are also described in the literature (Zimmermann et al. (2003) Vox Sangiuinis 85: 283-289; Martineau et al. (2004) Biomaterials 25: 4489-4502).

Preferably, the platelets are lysed by freezing and thawing them. Suitable temperatures for freezing the cells are from −20° C. to −196° C., preferably the temperature is −30° C. to −120° C. and most preferably the temperature is −80° C.

The platelet fragments produced by the lysis may be removed by centrifugation of the thawed platelet lysate to avoid an allo-immunization against platelet antigens. The supernatant may then be used for supplementing the medium. Suitable centrifugation conditions are e.g. 4000 g for 15 minutes.

Although the use of the medium of the present invention is described in detail for the culturing of MSCs, it is also suitable for culturing other cells, in particular cells the growth of which depends on growth factors released by the platelets, e.g. endothelial cells such as HUVEC, endothelial progenitor cells (EPC) and endothelial stem cells (ESC), fibroblasts, osteoblasts, keratinocytes, and cartilage cells.

Preferred cell types which may be cultured in a medium comprising the cell culture supplement in accordance with the invention are stem cells and progenitor cells.

The term “stem cells” refers to cells which have retained the capacity to proliferate and differentiate into different cell types. Stem cells in accordance with the present invention can comprise pluripotent and totipotent stem cells.

Totipotent stem cells still have the capacity to differentiate into all different cell lineages of a whole organism and thus may give rise to a complete organism. Plulipotent stem cells have retained the capacity to differentiate only into distinct cell lineages and cell types.

It is understood that the term “stem cells” in accordance with the invention does not comprise human embryos. Furthermore, it is understood that the term “stem cells” does not comprise pluripotent stem cells which have been directly derived from a human embryo. Embryonic stem cells which have been derived from publicly available and previously established stem cell lines are understood to fall within the meaning of the term “stem cells” as used by the present invention.

In a preferred embodiment, stem cells will be neural stem cells, mesenchymal stem cells, endothelial stem cells, haematopoietic stem cells or epithelial stem cells. A particularly preferred cell type are mesenchymal stem cells.

Mesenchymal stem cells (MSCs), which are also known as marrow stromal cells or mesenchymal progenitor cells, are defined as self-renewable, multi-potent progenitor cells with a capacity to differentiate into several distinct mesenchymal lineages. MSCs have been demonstrated to differentiate into lineage-specific cells that form bone, cartilage, fat, tendon and muscle tissue. Their surface phenotype has been defined as being negative for CD45 and CD34 and positive for CD73 (SH4), CD105 (SH2) and CD90 (Thy-1). They can be isolated from the bone marrow by their ability to adhere to the plastic substrate of the cell culture plate which distinguishes them from haematopoietic cells which do not adhere to the plate. Because most MSC populations lack specific cell surface markers, many isolation protocols are based on the process of negative selection meaning that cells lacking the expression of endothelial and hematopoietic cell markers are sorted out and maintained as MSC cultures.

The differentiation of MSCs to the chondrogenic lineage can be initiated by the treatment of the cells with TGF-β1 in a concentration of 10 ng/ml, whereas the osteogenic medium contains 100 nM dexamethasone, 10 mM β-glycerophosphate and 0.05 mM ascorbic acid-2-phosphate. Supplements for differentiating the MSCs to the osteogenic or the adipogenic lineage are also commercially available from Stem Cell, Inc.

The induction of the chondrogenic differentiation can be detected by staining the MSC derived chondrogenic cells with Safranin O after 4 weeks of incubation with chondrogenesis-inducing culture medium. Safranin O is a cationic stain that binds to cartilage glycosaminoglycans (GAG) such as chondroitin sulfate and keratan sulfate. The osteogenic differentiation can be detected by a positive reaction to alkaline phosphatase and von Kossa silver staining after 4 weeks of induction with medium containing osteo-inductive supplement.

The MSCs may be cultured in a medium supplemented with a supplement of the present invention for 6 to 15 days, preferably for 8 to 14 days, more preferably for 10 to 13 days and most preferably for 11 or 12 days. The expert knows that the cultivation time depends on the density of the cells when they are seeded. If the cells are seeded in a high density, they will be cultured for a shorter time than cells which are seeded in a low density. Usually the cultivation is stopped when the cells are subconfluent. “Subconfluent” means that the cells have not reached confluency, i.e they do not cover the entire surface of the cell culture vessel. When the cells are subconfluent, they cover at least 60% or 70%, preferably they cover at least 80% or 85%, more preferably they cover at least 90%, 92% or 94%, and most preferably they cover 96% or 98% of the surface of the cell culture vessel.

The MSCs cultured in a medium supplemented with the supplement of the present invention may be used in various ways. For example, MSCs can be used in bone healing and regeneration by loading scaffolds with the MSCs which serve then as a potential substitute for autologous and allogeneic bone grafts (Arinzeh (2005) Foot Ankle Clin. 10(4): 651-665). Furthermore, MSCs could be associated with biomaterials and implanted in pathological joints for use in osteoaithritis or rheumatoid arthritis (Jorgensen et al (2004) Curr Opin. Biotechnol. 15(5): 406-410). It has also been suggested to use mesenchymal stem cells in spine surgery (Helm and Gazit (2005) Neurosurg Focus 19(6): E13). Due to their immunomodulatory potential, MSCs may also be used in the treatment of autoimmune reactions such as collagenopathies, multiple sclerosis and graft versus host disease (reviewed in Krampera et al. (2006) Curr. Opin. Pharmacol. 6(4): 435-441). Finally, it has been suggested to use MSCs in cardiac regeneration after ischemia-induced death of cardiomyocytes (Minguell and Erices (2006) Exp. Biol. Med. 231(1): 39-49).

Besides culturing native, i.e. not genetically altered, MSCs, the cell culture medium supplement of the present invention may also be used for culturing genetically altered cells. Several strategies have been employed to deliver transgenes into (mesenchymal stem) cells, most of them using viral vectors such as oncogenic retroviruses, lentivirus-based vectors, adenoviral vectors and adeno-associated viruses. Non-viral methods for transgene delivery include electric field-induced molecular vibration, electroporation and liposome-based transfection. Methods of transgene delivery into MSCs are reviewed in Reiser et al. (2005) Expert Opin. Biol. Ther. 5(12): 1571-1584.

The choice of the transgene to be introduced into the MSCs depends on the intended use of the genetically altered cells. VEGF-expressing MSCs could be used for the treatment of myocardial infarction (Matsumoto et al. (2005) Arterioscler. Thromb. Vasc. Biol. 25: 1168-1173). Transduction of MSCs with bone morphogenetic proteins or transforming growth factors may be useful in osteogenesis, osteoinduction and osteochondral and cartilage repair (see for example Dayoub et al. (2003) Tissue Eng. 9(2): 347-356; Tsuda et al. (2003) Mol. Ther. 7(3): 354-365). MSCs expressing BDNF (brain-derived neurotrophic factor) or GDNF (glial cell line-derived neurotrophic factor) may be used to reduce the ischemic damage after stroke (Kurozumi et al. (2004) Mol. Ther. 9(2): 189-197; Kurozumi et al. (2005) Mol. Ther. 11(1): 96-104).

The “progenitor cells” are early descendants of stem cells which may lose their ability of self-renewal. The progenitor cells are able to differentiate to different cell types within one germ layer, but in contrast to stem cells they cannot differentiate to cells of a different germ layer. There are three different germ layers, i.e. endoderm, ectoderm and mesoderm which are formed by gastrulation. The endoderm is the internal cell layer of the embryo from which the lung, digestive tract, bladder and urethra are formed. The ectoderm is the surface layer of the embryo that develops into the epidermis, skin, nerves, hair, etc. The mesoderm is the middle cell layer of the embryo from which the connective tissue, muscles, cartilage, bone, lymphoid tissues, etc. are formed.

Within the context of the present invention, the progenitor cells are preferably endothelial progenitor cells.

The invention is based on the surprising finding that a plasma-free platelet lysate stimulates the proliferation of cells and preferably of MSCs even better than a platelet lysate containing plasma and therefore may be used as a cell culture medium supplement avoiding the potential negative effects of plasma. A further advantage is that the plasma-free platelet lysate does not raise concerns about blood group compatibility.

The invention is further illustrated by the following examples, which should not be construed as limiting. The contents of all references, patent applications, patents, published patent applications, tables and appendices cited throughout this application are hereby incorporated by reference.

### EXAMPLES

### 1) Preparation of a Cell Culture Supplement Comprising Plasma-Free Platelet Lysate and Human Serum Albumin

The donated whole blood (450 ml) was anticoagulated by addition of 62 ml CPD (26.3 g/l sodium dihydrate, 3.27 g/l citric acid monohydrate, 25.5 g/l glucose monohydrate and 2.51 g/l sodium dihydrogenphosphate dihydrate). After a resting period of 16 hours at 22±2° C. the blood units were centrifuged at 4247 g at 22° C. for 10 minutes. Erythrocytes and plasma were separated automatically (Compomat G3, NPBI, Amsterdam, The Netherlands) from the BC fraction and transferred into satellite containers. Randomised buffy coats from four different ABO- and Rhesus-identical donations and one bag containing plasma from one of the four donors were connected sterilely (TSCD, Terumo Corp., Tokyo, Japan) in series and pooled by gravity in the lowest container. The pooled BCs were centrifuged at 341 g at 22° C. for 6 minutes and the platelet-rich plasma was leukocyte-depleted by inline filtration (PALL Autostop, Pall, Dreieich, Germany) and transferred into a platelet storage bag (ETX, PALL, Dreieich, Germany). The mean±SD platelet concentration was 0.93±0.10×10⁹/ml.

To remove the plasma the pooled concentrate was centrifuged at 2500 g for 10 minutes after addition of 10% ACD-A to avoid aggregate formation and the supernatant was discarded. A solution containing 5% human albumin (Immuno Baxter AG, Vienna, Austria) and 10% ACD-A was added to the pellet and the platelet pellet was resuspended to yield a final platelet concentration of 1−2×10⁹/ml. The suspension of platelets was frozen at −80° C. for lysis of the platelets and release of growth factors. After thawing, several units of platelet lysate were pooled to avoid individual donor variations and the pool was frozen again at −80° C. until use.

### 2) Proliferation Experiments

MSCs derived from bone marrow (passage 3) were used for in vitro expansion in alpha-modified minimal essential medium (a-MEM) supplemented with 10% US defined FBS (HyClone, Logan, USA), 10% FBS selected by the European Group for Blood and Marrow Transplantation (EBMT) for clinical studies (EBMT FBS; HyClone), 10% platelet lysate with plasma (PL) or 10% plasma-free platelet lysate in human albumin solution (PL-HA). Cells were seeded in a density of 50-100 cells/cm², medium was changed twice weekly. On day 12 cells were harvested using 0.25% trypsin/1 mM EDTA and the number of living cells was calculated after staining the cells with Trypan blue which stains the dead cells. All cell numbers given refer to the number of living cells.

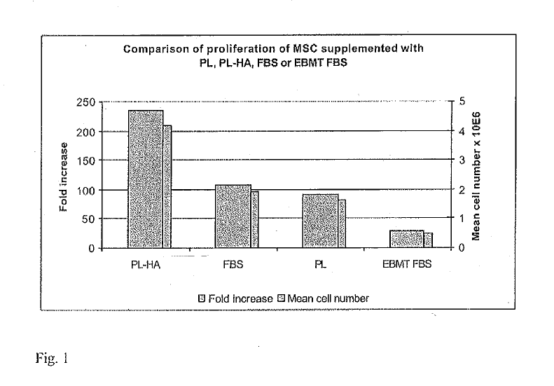

FIG. 1 shows that the mean cell number and the fold increase (FI) of initially seeded MSCs was highest in culture supplemented with PL-HA (4.71×10⁶; FI 209.27) compared to FBS (2.15×10⁶; FI 95.59), PL (1.81×10⁶; FI 80.59) and EBMT FBS (0.58×10⁶; FI 26.04). In this particular experiment replacement of plasma by human albumin solution led to a more than twice as high proliferation of MSCs than in FBS- and PL-supplemented culture and to an eight fold as high proliferation rate of MSCs than in cultures stimulated with EBMT FBS.

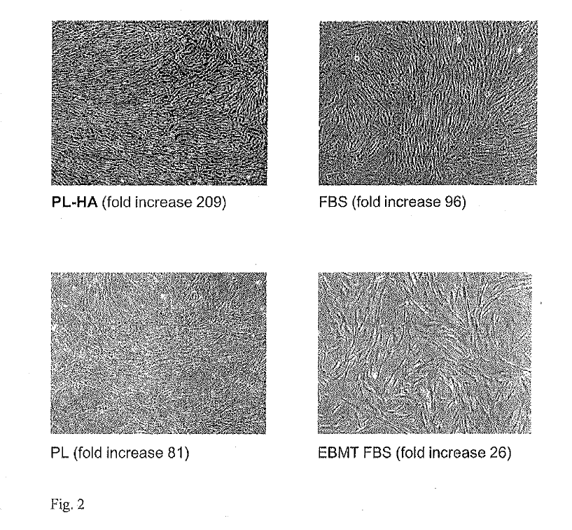

In FIG. 2 the morphological features of MSCs expanded by the four different supplements are shown. It is obvious that MSC colonies stimulated by PL-HA grow more tightly than in the other media and reach a higher cell density consistent with the larger cell quantity produced as shown in FIG. 1 without losing their characteristic shape.

### 3) Cytokine and Growth Factor Assays

Cell culture media were frozen before use on day 0 and cell culture supernatants were frozen on day 12 at −70° C. Multiplex human cytokine detection (22-plex®, Upstate, Lake Placid, N.Y.) was used to measure the production of GM-CSF, IL-2, IL-2R, IL-5, IL-6, IL-7, 1L-8, IL-10, 1L-13, IP10, TNF-A, MCP-1 and RANTES in the supernatants of the platelet derived supplements. Additionally, growth factors as EGF, FGF-2, Flt-3 ligand, PDGF-AA, PDGF-BB and VEGF were measured (Beadlyte® Human Growth Factor Beadmaster™ Kit, Upstate, Lake Placid, N.Y.).

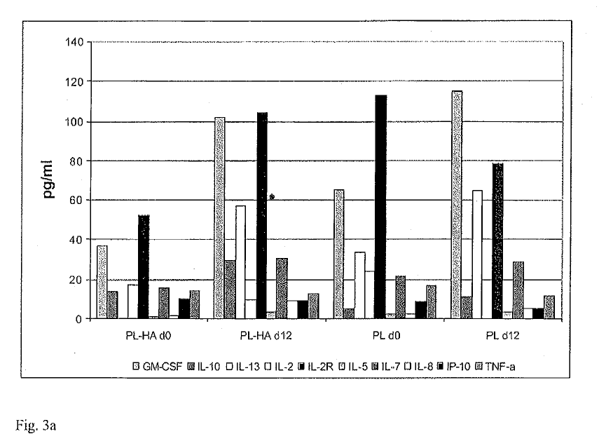

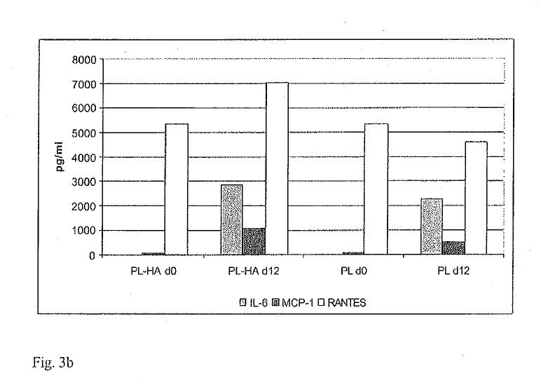

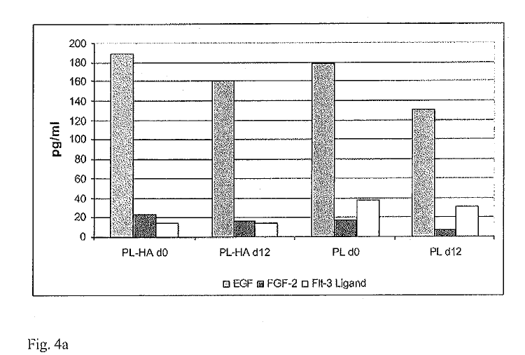

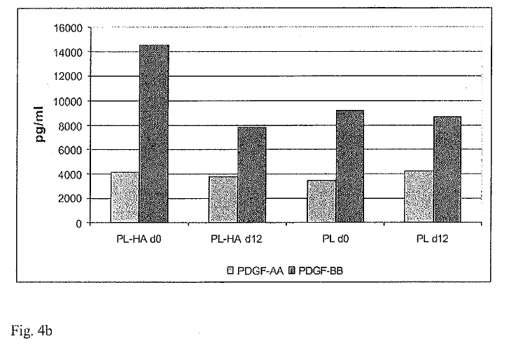

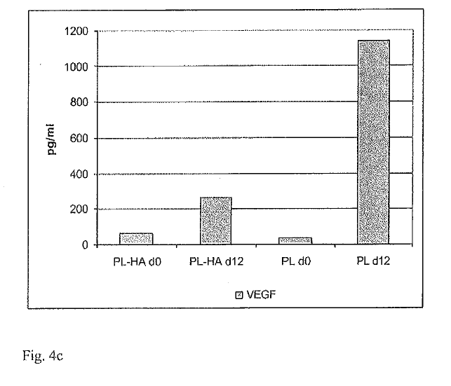

Results of measured cytokines and growth factors in the supernatants of cultures with PL-HA and PL on day 0 and day 12 are demonstrated in FIGS. 3 and 4. The tendency of GM-CSF, IL-6, IL-7, IL-13 and MCP-1 levels to increase during 12 days of culture can be observed in PL-HA-as well as in PL-supplemented cultures. In contrast, levels of EGF and PDGF-BB decrease during proliferation of MSCs in both culture systems indicating consumption of the stimulating factors. VEGF production by MSCs was more obvious in PL compared to PL-HA which may be of importance for vascular regenerative therapy.

1. Cell culture medium supplement comprising plasma-free platelet lysate.

2. Supplement according to claim 1, further comprising a substance selected from the group consisting of albumin, dextran and hydroxyethyl starch.

3. Supplement according to claim 2, wherein the substance is albumin.

4. Supplement according to claim 2 or 3, wherein the albumin is human serum albumin.

5. Supplement according to any of claims 2 to 4, wherein the albumin is present in a concentration of 2-7% v/v, preferably 5% v/v.

6. Supplement according to any of the preceding claims, further comprising acid citrate dextrose or citrate phosphate dextrose.

7. Supplement according to any of the preceding claims, wherein the lysate is prepared from a solution with a platelet concentration of 1×10⁸ to 5×10⁹ per ml, preferably from a concentration of 1-2×10⁹ per ml.

8. Cell culture medium supplemented with the supplement of any of claims 1 to 7.

9. Medium according to claim 8, wherein the supplement is present in a concentration of 1-20% v/v, preferably 10% v/v.

10. Medium according to claim 8 or 9, the medium being α-MEM.

11. Method for preparing the supplement according to any of claims 1 to 7, comprising the following steps:
a) preparing platelet-rich plasma;
b) removing the plasma; and
c) lysing the platelets.

12. Method according to claim 11, further comprising the step of adding a substance selected from the group consisting of albumin, dextran and hydroxyethyl starch before or after lysing the platelets.

13. Method according to claim 12, wherein the substance is albumin.

14. Method according to claim 12 or 13, wherein the albumin is added to yield a final concentration of 2-7% v/v, preferably of 5% v/v.

15. Method according to any of claims 11 to 14, wherein the platelet-rich plasma is prepared from buffy coat units.

16. Method according to claim 11 to 15, wherein the plasma is removed by centrifugation.

17. Method according to any of claims 11 to 16, wherein the platelets are lysed by freezing and thawing them.

18. Method according to any of claims 11 to 17, wherein the concentration of the platelets before lysis is 1×10⁸ to 5×10⁹ per ml, preferably 1-2×10⁹ per ml.

19. Method for preparing the medium according to any of claims 8 to 10, comprising the step of mixing a cell culture medium with the supplement according to any of claims 1 to 7.

20. Method of culturing cells, wherein the cells are cultured in a medium according to any of claims 8 to 10.

21. Method according to claim 20, wherein the cells are mesenchymal stem cells or progenitor cells.
